(19) United States
(12) Patent Application Publication
(10) Pub. No.: US 2026/0070951 A1
(43) Pub. Date: Mar. 12, 2026

(54) MICROPAKINE K2 ANTIMICROBIAL POLYPEPTIDE, COMPOSITION COMPRISING IT AND USE FOR TREATING BACTERIAL INFECTIONS

(71) Applicant: Universidad Autónoma de Chile, Santiago (CL)

(72) Inventors: Gino CORSINI, Santiago (CL); Paz ORELLANA, Santiago (CL); Guillermo WIESE, Santiago (CL); Alequis PAVÓN, Santiago (CL); Lorena SALAZAR, Santiago (CL); Ana GUTIERREZ, Santiago (CL); Manuel GIDEKEL, Santiago (CL)

(73) Assignee: Universidad Autónoma de Chile, Santiago (CL)

(21) Appl. No.: 19/252,558

(22) Filed: Jun. 27, 2025

Related U.S. Application Data
(63) Continuation of application No. PCT/CL2022/050144, filed on Dec. 29, 2022.

(51) Int. Cl. C07K 14/21; A01N 63/50; A01P 1/00; A61K 38/00; A61P 31/04

(52) U.S. Cl. CPC C07K 14/21; A01N 63/50; A01P 1/00; A61P 31/04; A61K 38/00

(57) ABSTRACT

A polypeptide corresponding to the amino acid sequence SEQ ID No 1, wherein said polypeptide has antimicrobial activity; An antimicrobial composition, comprising said polypeptide, and optionally a second antimicrobial agent and a carrier; Use of the antimicrobial composition to inhibit the growth and proliferation of Gram-positive and Gram-negative bacteria.

### CROSS-REFERENCE TO RELATED APPLICATION

This application is a continuation application under 35 U.S.C. 111 (a) of International Patent Application PCT/CL2022/050144 filed on Dec. 29, 2022, and designated the U.S., the entire contents of which are incorporated herein by reference.

The contents of the Sequence Listing XML file are herein incorporated by reference. The name of the Sequence Listing XML file is “micropac2.xml.” The date of creation of the Sequence Listing XML file is Dec. 29, 2022. The size of the Sequence Listing XML file in bytes is 1,988 bytes.

### SPECIFICATION

The present invention is related to the field of molecular biology and biotechnology applied to the use of bacterial peptides that have bactericidal or bacteriostatic properties on different microorganisms.

### BACKGROUND

In recent decades, the indiscriminate use of antibiotics has led to an unresponsiveness of pathogens that cause bacterial infections in humans, leading to resistance, which currently represents a serious public health problem worldwide. The availability of new antimicrobial molecules for therapeutic use has decreased considerably in the face of the sustained increase in bacterial resistance to various antibiotics used in clinical use, leading to the selection of multi-resistant pathogenic bacteria, such as Staphylococcus aureus resistant to methicillin and vancomycin (Mandal et al., 2015; Fussen et al., 2015; Ostojic et al., 2015) and even environmental bacteria (Calisto et al., 2021, Orellana et al., 2022).

The rapid and effective resistance displayed by this type of bacteria has limited the use of many currently available antibiotics, becoming a growing concern for public health organizations. Faced with this complex situation, various initiatives have been carried out by scientists worldwide, which have led to the development, by Infectious Diseases Society of America (IDSA), the program called ESKAPE, which considers resistant bacteria Enterococcus faecium, Staphylococcus aureus, Klebsiella pneumoniae, Acinetobacter baumannii, Pseudomonas aeruginosa and Enterobacter spp. This acronym describes pathogens capable of “escaping” the action of antibiotics, representing new paradigms in relation to bacterial pathogenesis, transmission, and resistance (Rice, 2008). Therefore, ESKAPE pathogens are increasingly relevant to the search for antimicrobial therapy, leading to innovative strategies for the development of new antimicrobial options (Hijazi et al., 2018; Tommasi et al., 2015; Pendleton et al., 2013; Llaca-Díaz et al., 2012).

### Current Antimicrobial Development.

Currently, although more than 23,000 antibiotics have been discovered from different microorganisms (Manivasagan et al., 2014), only five new antimicrobials approved for use and application in patients with infections caused by clinically relevant bacterial agents have entered the market since 1980. However, as mentioned above, the indiscriminate use of currently existing therapeutic agents has led to bacterial pathogens that cause human infections not responding to this type of treatment, generating resistance. Worldwide, antimicrobial resistance poses a serious and growing threat to public health. It is a growing problem that involves new bacterial species and new resistance mechanisms every day. This phenomenon, observed in microbiology laboratories, represents a clinical problem and hinders the proper management of patients suffering from different infectious pathologies (Valenzuela et al., 2003). In this sense, the current development of new antimicrobials has not allowed us to respond to the increase in bacterial resistance to multiple antibiotics, even in bacteria that today have been reported to be resistant to all available antibiotics, as is the case of the so-called “killer bacteria” Klebsiella pneumoniae, a pan-resistant bacteria reported in Nevada, USA, where antimicrobial susceptibility testing indicated that the isolate was resistant to 26 antibiotics, including aminoglycosides, polymyxins, tigecycline (a tetracycline derivative developed in response to emerging antimicrobial resistance), and colistin (Chen et al., 2017). Thus, a significant gap has emerged between the generation of new antimicrobial compounds and the rise in resistance, leading to a latent threat in the face of the increasing number of patients at risk with bacterial infections (Boucher et al., 2009).

The development and discovery of new antimicrobials has become more complicated in recent years due to regulations governing the approval of new drugs by the Food and Drug Administration (FDA), competition between pharmaceutical companies and the economic and scientific cost involved (Shlaes et al., 2013; Wright et al., 2014). However, despite the difficulty of this subject, it is imperative to continue the search for new antimicrobials, because science also has a social mission to improve people's quality of life, ultimately preventing thousands of patients from dying from bacterial infections (Cooper and Shlaes, 2011).

### Antimicrobial Peptides.

The bacterial antagonism given by antimicrobial peptides has gained importance due to the high toxicity effect they present against a wide spectrum of bacteria and the low resistance generated (Maqueda et al., 2008). Various microorganisms present in nature produce antimicrobial peptides, which in natural conditions they use as a mechanism of adaptation and competition against other microorganisms, inhibiting their growth and thus favoring their establishment in the bacterial ecosystem (Jenssen et al., 2006; Calisto et al., 2021).

Recent research has focused on the isolation and characterization of bacteria that inhabit extreme environments, specifically the Antarctic continent, because (described below) it is considered a large reservoir of bacterial biodiversity (Texeira et al., 2010). Bacteria that grow in these environments compete to maintain an ecological niche, releasing molecules that inhibit or kill other microorganisms. Specifically, these peptides, polypeptides or proteins with antimicrobial properties secreted by bacteria are called bacteriocins (Etayash et al., 2016; Riley et al., 2002; Nissen-Meyer, 1997).

### Bacteriocins.

Bacteriocins have a variable molecular mass and have different mechanisms of action, levels of activity and physicochemical properties (Stoyanova et al., 2012). These compounds are synthesized by both Gram-positive and Gram-negative bacteria (Jeevaratnam et al., 2005).

In Gram-positive bacteria, bacteriocins are structured by 20 to 60 amino acids that exhibit high bactericidal or bacteriostatic activity against related bacterial strains and pathogenic microorganisms that damage food (Chen and Hoover, 2003). They possess disulfide bridges, thioether or thiol groups, and are produced during the exponential phase of bacterial growth (Cotter et al., 2005), are highly stable at high temperatures due to the formation of stable crosslinks and hydrophobic regions (Beristain-Bauza et al., 2012; Alquicira, 2006; Chen and Hoover, 2003; Jack et al., 1995). Furthermore, bacteriocins are inactivated by some proteases, a characteristic that allows them, due to their protein nature, to be harmless to humans (Beristain-Bauza et al., 2012; Quintero, 2006). They have a broad spectrum of antimicrobial action, showing inhibition, at concentrations lower than 10 ppm, against other Gram-positive and Gram-negative pathogenic bacteria such as Clostridium botulinum, Enterococcus faecalis, Listeria monocytogenes, Staphylococcus aureus, Salmonella enterica, Escherichia coli and some species of Bacillus, although greater inhibition has been shown against Gram-positive bacteria (Jeevaratnam et al., 2005; Wu et al., 2004).

The synthesis of bacteriocins is encoded in genes organized in operons, which can be present in transposons, plasmids or in the bacterial chromosome (Altena et al., 2000; Engelke et al., 1992). Its synthesis depends on the genetic structure, composed of:
A structural gene encoding a pre-bacteriocin, which consists of an amino-terminal signal sequence that allows stabilization during translation, preventing the bacteriocin from becoming activated inside the producing bacteria. It also provides a recognition signal for the transport system and maintains the conformation of the pre-bacteriocin.
An immunity gene, which allows the translation of a protein that confers protection to the bacteria against the antimicrobial effect of the bacteriocin itself and is closely linked to the structural gene, forming part of the same transcription unit. It is important to note that each bacteriocin has its own immunity protein, which is expressed alongside it. However, the sequence identity between immunity proteins is surprisingly low considering the homology that bacteriocins have with each other (Aymerich et al., 1996). Hydrophobicity analyses of some immunity proteins revealed that they possess possible transmembrane segments, which would indicate that these factors are located in the target of action of most bacteriocins, thus carrying out “in situ” protection against the antimicrobial action itself (Fremaux et al., 1998).
A gene encoding an ABC transporter that excretes bacteriocin and catalyzes its processing (Rebuffat, 2013; Nes et al., 1996; Harvard et al., 1994; Klaenhammer, 1993). The gene encoding the ABC transporter may be part of the same operon as the bacteriocin or a different operon.

The consensus elements found in the signal peptide region are characterized by two glycine residues adjacent to and prior to the processing site and domains of hydrophobic and hydrophilic residues separated by conserved distances. Transporters that recognize double glycine (GG) type signal peptides contain three domains, which may be encoded in a single polypeptide or in separate polypeptides (Rebuffat, 2013).

In the specific case of microcin type bacteriocins, these are found in a cluster of genes present in a plasmid or in the bacterial chromosome, which code for the precursor of microcin, the immunity gene, the proteins that participate in secretion (FIGURE *1) and in some cases, enzymes that participate in post-translational modifications (Duquesne et al., 2007; Thomas et al., 2004).

In general, class I microcins (MccB17, MccC7/C51 and MccJ25) are encoded by a gene cluster, where the autoimmunity gene is located far from the structural microcin gene. Adjacent to the microcin gene are two or three genes involved in post-translational modification and a gene involved in both export and autoimmunity (Duquesne et al., 2007).

In the case of class II microcins, they are characterized by having at least two genes involved in export. These genes, which are homologous within this class of microcins, require to be functional the product of the tolC gene, which is provided by the host. The genetic system of class Ila microcins is composed of four genes, organized into two operons, one for the microcin and immunity system and the other for the export system (Duquesne et al., 2007). The genetic system of class IIb microcins is encoded in the bacterial chromosome, presenting a complex transcriptional organization, with genes involved in post-translational modification (Duquesne et al., 2007).

### Applications of Bacteriocins.

Bacteriocins have various applications in both humans and animals, one of the most important being their potential antibiotic use against microorganisms and as an alternative to currently used antibiotic therapies that are less effective due to the resistance that has been generated in pathogenic strains (Riley and Wertz, 2002). They differ from traditional antibiotics because they act on a narrow spectrum of bacteria that are related to the producing strain (Riley and Wertz, 2002). Furthermore, bacteriocins are important in the food industry, where they have been used as biopreservatives and prebiotics, among other uses (Gillor et al., 2008; Ross et al., 2002), but also in medical and veterinary applications (Table 1) (Pieterse et al., 2010).

TABLE 1
Potential medical and veterinary applications of some
bacteriocins. Adapted from Pieterse et al., 2010.

| Bacteriocins by Gram- | | |
|---|---|---|
| negative bacteria. | Producer | Potential use |
| Microcins J25 and | E. coli | Treatment of E. coli infections and |
| N (Ex 24) | | Salmonella in chickens. |
| Microcin E492 | E. coli | Antiproliferative of tumor cells. |
| Colicins E1, E4, E7, | E. coli | Treatment of hemorrhagic colitis and |
| E8, K and S4 | | hemolytic uremic syndrome (HUS) |
| | | caused by E. coli O157:H7. |

### Pseudomonas and Production of Antimicrobial Molecules.

As mentioned above, several studies have described the genus Pseudomonas as one of the most diverse and ecologically significant bacterial groups on the planet. Members of this genus have been isolated from different environments, such as terrestrial, aquatic, marine, associated with plants, animals, and, as described above, the Antarctic continent (Orellana et al., 2017; Matthijs et al., 2014; Spiers, 2000). This universal distribution suggests a wide range of physiological and genetic adaptability, allowing a remarkable capacity to produce, for example, antimicrobial molecules. Further acquisition and analysis of whole genome sequences of this bacterial genus provides relevant information on the phenotypic characteristics of Pseudomonas isolates. Meanwhile, recent studies carried out on microorganisms from the Antarctic continent allowed sequencing the genome of the bacteria Pseudomonas sp., an Antarctic isolate from the plant D. antarctica Desv. (Orellana et al., 2017).

The genus Pseudomonas, in particular the species P. aeruginosa, is recognized for the production of high molecular mass proteins, with antimicrobial activity on other species of Pseudomonas, called Pyocins (Higerd et al., 1987; Sano and Kageyama, 1984). There are three types of pyocins described to date:
R-type pyocins are inflexible proteins that form a multiprotein complex, similar to the contractile structure of a bacteriophage. They exert their function by forming a pore or channel in the cytoplasmic membrane, causing its depolarization (Michael-Briand and Baysse, 2002).
S-type pyocins are similar to colicins. They are composed of a large protein and a smaller protein that are held together. The larger protein has antimicrobial activity, while the smaller protein acts as an immune barrier against the producing strain (Michael-Briand and Baysse, 2002).
F-type pyocins are proteins with a flexible structure and a function similar to R-type pyocins.

Among the bacteriocin-type antimicrobial molecules, the LlpA protein has been described, which has a molecular mass of 30 kDa and is secreted by the Pseudomonas sp. BW11M1 strain. It has antimicrobial activity against Pseudomonas putida strains. (Parret et al., 2002). Furthermore, the PsVp10 protein with a molecular mass of 2.5 kDa, has demonstrated antimicrobial activity on Gram-positive and Gram-negative bacteria (Padilla et al., 2002; Hubert et al., 1998).

WO2020187822A1 describes the genome sequence of Pseudomonas sp. CECT8708 (SEQ1), which contains the sequence of an open reading frame (ORF) with a similarity of 91.1% with respect to the Sequence of the present invention, also describes the uses of the bacteria in the control of bacterial and/or fungal infections in a plant. However, it does not mention using the ORF included in its SEQ 1, to specifically combat: Escherichia coli, Klebsiella pneumoniae, Salmonella enterica serovar Typhi, nor other Enterobacteriaceae.

Document US20200178540A1 describes a sequence (SEQ 1) with a similarity of 83.3% to the Sequence of the present invention. It describes biopesticides that are active against Erwinia species, particularly against the fire blight phytopathogen, Erwinia amylovora. Also describes their use as a biological control agent against fire blight. However, it does not mention using its SEQ 1 to specifically combat Escherichia coli, Klebsiella pneumoniae, Salmonella enterica serovar Typhi, nor other Enterobacteria.

Document WO2022184525A1 describes a sequence (SEQ2) with a similarity of 84.3% to the Sequence of the present invention. It describes its use in the field of bioremediation and metal recovery. However, it does not mention using its SEQ 2 to specifically combat Escherichia coli, Klebsiella pneumoniae, Salmonella enterica serovar Typhi, nor other Enterobacteria.

Document US20190191708A1 describes a sequence (SEQ8) with a similarity of 89.1% to the Sequence of the present invention. It describes bacterial strains effective in controlling, treating or preventing infection of solanaceous plants with Phytophthora infestans. Biopesticide formulations containing one or more of the bacterial strains or extracts thereof are also provided, as well as the use of the bacterial strains, extracts, or biopesticide formulations in the control, treatment, and/or prevention of potato late blight. However, it does not mention using its SEQ 8 to specifically combat: Escherichia coli, Klebsiella pneumoniae, Salmonella enterica serovar Typhi, nor other Enterobacteria.

U.S. Pat. No. 8,945,540B2 describes compositions for enhancing the antibacterial activity of myeloperoxidase and methods of using them. It describes their highly effective use in inhibiting both Gram-positive and Gram-negative organisms, such as Enterococcus faecalis, Enterococcus faecium, Staphylococcus aureus, Staphylococcus epidermidis, Streptococcus agalactiae, Streptococcus Group C, Streptococcus Group F, Streptococcus Group G, Streptococcus pyogenes, Citrobacter freundii, Enterobacter cloacae, Escherichia coli, Klebsiella pneumoniae, Proteus mirabilis, Acinetobacter spp., Pseudomonas aeruginosa, Aeromonas hydrophilia and Pasteurella multocida, Bacillus spp. and Clostridium spp., Aspergillus spp., Fusarium spp., Trichophyton spp. However, it only describes chemical components, with chemical action; the use of proteins, peptides, or enzymes with antibiotic action is not described.

### DESCRIPTION OF THE INVENTION

### Technical Problem

The present invention seeks to provide antimicrobial molecules that combat infection by growth of Gram-positive and Gram-negative bacteria, human pathogens such as: Escherichia coli, Klebsiella pneumoniae, Salmonella enterica serovar Typhi among other enterobacteria.

The present invention describes an antimicrobial polypeptide, comprising the amino acid sequence SEQ ID No 1:

ALSLSALTTTAHAADDMQKCFGVAEAGKNDCAAGAGTSCAGTSKVKDQA
NAWKLVPAGTCLKTPSATSPTGFGQEAAFTAKS;

where said polypeptide is secreted and has antimicrobial activity.

The present invention also describes an antimicrobial composition, comprising said antimicrobial polypeptide of SEQ ID 1; and optionally a second antimicrobial agent and a carrier.

The present invention describes that in said antimicrobial composition, the vehicle is selected from: an organic solvent, aqueous solvent, non-aqueous solvent, a pH buffer and distilled water.

The present invention describes that in said antimicrobial composition, the polypeptide of amino acid sequence SEQ ID 1 is found in a concentration of 100-2000 AU/mL.

The present invention describes that in said antimicrobial composition, the polypeptide of amino acid sequence SEQ ID 1 is found at a concentration of 200 AU/mL.

The present invention describes that in said antimicrobial composition, the second antimicrobial agent is selected from the group of bacteriocins, beta-lactam antibiotics and tetracycline-type antibiotics.

The present invention also describes the use of said antimicrobial composition because it serves to inhibit the growth and proliferation of Gram-positive and Gram-negative bacteria.

The present invention describes that said use, serves to inhibit the growth of bacteria selected from the group consisting of E. coli, E. coli O157:H7, Vibrio parahaemolyticus, Citrobacter freundii, Salmonella enterica, Shigella flexneri, Shigella boydii, Shigella sonnei, Klebsiella oxytoca, Klebsiella pneumoniae and Pseudomonas aeruginosa among other Gram-negative bacteria.

The present invention describes that, in said use, the second antimicrobial agent is selected from the group of bacteriocins, beta-lactam antibiotics and tetracycline-type antibiotics. Said use serves to inhibit the growth of bacteria selected from the group consisting of E. coli, E. coli O157:H7, Vibrio parahaemolyticus, Citrobacter freundii, Salmonella enterica, Shigella flexneri, Shigella boydii, Shigella sonnei, Klebsiella oxytoca, Klebsiella pneumoniae, Pseudomonas aeruginosa, Morganella morganii, Proteus mirabilis, Proteus vulgaris, Staphylococcus aureus, Staphylococcus epidermidis, Staphylococcus saprophyticus, Enterobacter sp., Streptococcus pneumoniae, Streptococcus pyogenes, Streptococcus mutans, Enterococcus faecium, Enterococcus faecalis, Listeria monocytogenes, Lactobacillus spp., Lactococcus spp., Bacillus cereus, Bacillus subtilis and Cutibacterium acnes among other Gram-negative and Gram-positive bacteria.

The present invention describes the use of said composition, because it serves for the preparation of a medicine useful in the treatment of conditions and/or diseases caused by Gram-positive and Gram-negative bacteria.

The present invention describes the use of said composition, because it serves in the preparation of a medicament useful in the treatment of bacteria selected from the group consisting of: E. coli, E. coli O157:H7, Vibrio parahaemolyticus, Citrobacter freundii, Salmonella enterica, Shigella flexneri, Shigella boydii, Shigella sonnei, Klebsiella oxytoca, Klebsiella pneumoniae, Pseudomonas aeruginosa, Morganella morgani, Proteus mirabilis, Proteus vulgaris, Staphylococcus aureus, Staphylococcus epidermidis, Staphylococcus saprophyticus, Enterobacter sp., Streptococcus pneumoniae, Streptococcus pyogenes, Streptococcus mutans, Enterococcus faecium, Enterococcus faecalis, Listeria monocytogenes, Lactobacillus spp., Lactococcus spp., Bacillus cereus, Bacillus subtilis and Cutibacterium acnes among other Gram-negative and Gram-positive bacteria.

In the present invention, bacteria that inhabit extreme environments were isolated and characterized, specifically in the Antarctic continent, which is considered a major reservoir of bacterial biodiversity. Bacteria that grow in these environments compete to maintain an ecological niche, releasing molecules that inhibit or kill other microorganisms.

Specifically, SEQ ID 1 corresponds to a polypeptide sequence that forms an antimicrobial molecule with an inhibitory effect on human pathogenic bacteria. This SEQ ID 1 was obtained from the Antarctic bacteria K2115 (BA K2115), isolated from the rhizosphere of Deschampsia antarctica. For this purpose, BA K2115 was characterized based on morphology, Gram staining, enzymatic activity, antimicrobial susceptibility, and the 16S rRNA gene was sequenced. The antimicrobial action spectrum of BA K2115 on human pathogenic bacteria was determined. Its genome was sequenced, and the genetic elements involved in the synthesis and secretion of an antimicrobial molecule that inhibited the growth of pathogenic bacteria were identified. Finally, the molecule with bacteriocin-type antimicrobial activity and its effect on human pathogenic bacteria were characterized. The biochemical and molecular results allowed us to identify BA K2115 as a new bacteria belonging to the Pseudomonas genus that exhibits antimicrobial activity against Gram-negative pathogenic bacteria.

In this way, the present invention contributes to the biotechnological development of antimicrobials, through the identification and characterization of a bacteriocin-type protein molecule secreted by an Antarctic bacteria, which serves as a potential alternative treatment against antibiotic-resistant human pathogens in infectious diseases of bacterial origin.

The present invention describes the molecule of sequence SEQ ID 1, obtained from the Antarctic bacteria Pseudomonas sp. K2115, which was isolated from the rhizosphere of Deschampsia Antarctica. This molecule inhibits the growth of human pathogenic bacteria through bacteriocin-type antimicrobial activity.

### EXAMPLES

### Example 1

Identification of Antarctic bacteria with antibacterial activity against human pathogenic bacteria.

From the 55 Antarctic bacterial isolates, the K2115 bacteria was identified, which had a growth inhibitory effect on Gram-negative bacteria such as E. coli, but not against the Gram-positive bacteria S. aureus tested.

The K2115 bacteria was identified and characterized using phenotypic and molecular methods, in order to establish the metabolic and genetic characteristics that allowed the bacteria to be identified at the genus level.

### Phenotypic Identification Methods.

The K2115 BA was characterized based on colony color and texture, bacterial morphology, Gram staining (Bartholomew and Mittwer, 1952), and optimal growth temperature, determined at 4° C., 18° C., and 37° C. As shown in Table 2, the K2115 bacteria presented a white colony with a mucoid texture and growth at 4° C. and 18° C., but not at 37° C., characteristics of a psychrophilic bacteria. The cell morphology corresponded to a short Gram-negative bacillus (Table 2).

TABLE 2
Phenotypic characterization of BA
K2I15 isolated from Antarctic soil.

| | bacteria | |
|---|---|---|
| | | Pseudomonas |
| Feature | K2I15 | aeruginosa (control) |
| Colony color | White | White (slight green tone) |
| Texture of the colony | Mucoid | Mucoid |
| Growth at 4° C. | + | − |
| Growth at 18° C. | + | − |
| Growth at 37° C. | − | − |
| Gram stain | Gram-negative | Gram-negative |
| Cell morphology | Short bacillus | Short bacillus |

+ Positive result
− Negative result

### Example 2

### Identification by Biochemical Tests.

To identify BA K2115, its main metabolic characteristics were established using conventional biochemical tests, which were performed considering incubation temperatures of 4° C. and 18° C. The bacteria presented a non-fermentative metabolism, mobility, grew in Simmons citrate medium and tested positive for the urease test at both incubation temperatures (Table 3). These results are characteristic of a non-fermenting bacteria of the genus Pseudomonas.

To complement these results, the API 20NE system was used, which determined the identification code 0046755 for BA K2115, corresponding to the bacteria Pseudomonas fluorescens with 84.1% identity. This result allowed us to suggest with greater certainty that BA K2115 corresponds to a bacteria belonging to the genus Pseudomonas.

TABLE 3
Biochemical tests of BA K2I15.

| | | | Pseudomonas | |
|---|---|---|---|---|
| | K2I15 | | aeruginosa (control) | |
| Biochemical test | 4° C. | 18° C. | 18° C. | 37° C. |
| TSI | + | + | − | + |
| Tilt | − | − | − | − |
| Bottom | − | − | − | − |
| H2S Gas Production | − | − | − | − |
| Fermentation of sugars: | − | − | − | − |
| Glucose, | − | − | − | − |
| Lactose, | − | − | − | − |
| Sucrose | − | − | − | − |
| Mobility | + | + | − | + |
| Indole test | + | + | − | + |
| Urea hydrolysis | + | + | − | + |
| Growth on Simmons citrate | + | + | − | + |

+ Positive result,
− Negative result

In order to genetically identify the K2115 bacteria, the 16S rDNA gene was amplified by PCR from DNA extracted from the bacteria.

The sequencing of the 16S rDNA gene resulted in a sequence of 1481 bp, which was analyzed by BLAST based on the comparison with sequences present in the GenBank database, which allowed the identification of BA K2115 as Pseudomonas sp., belonging to the γ-Proteobacteria Family, with 98% identity (Table 4).

TABLE 4
Identification of the Antarctic bacteria K2I15 based
on sequencing of the 16S rDNA gene and analysis
of the sequences by local BLAST alignment.

| | | | | % |
|---|---|---|---|---|
| | | | ID (Genus and/ | iden- |
| BA | Class | Family | or species) | tity |
| K2I15 | γ - Proteobacteria | Pseudomonaceae | Pseudomonas sp. | 98 |
| TI12 | γ - Proteobacteria | Pseudomonaceae | Pseudomonas sp. | 100 |

### Example 3

The antagonistic effect of BA K2115 on the pathogenic bacteria K. oxytoca, K. pneumoniae, S. Typhi, S. boydii and A. baumannii by the plate antagonism method. The test was carried out in the following media: Nutrient agar (Nut), Nutrient agar at one-third of its formulation (Nut ⅓), LB agar and LB agar at one-third of its formulation (LB ⅓) and three different incubation temperatures were considered: 4° C., 18° C. and 37° C. The results showed that the conditions where the greatest zone of growth inhibition of the indicator strain was obtained was in LB agar culture medium at 18° C. Therefore, this culture condition of the BA K2115 was selected for subsequent experiments. (Table 5; FIG. 1).

TABLE 5
Detection of bacterial antagonism in different culture conditions.

| | Temperature (° C.) | | |
|---|---|---|---|
| Culture medium | 4 | 18 | 37 |
| Nutrient Agar | + | + | − |
| Nutrient Agar ⅓ | + | + | − |
| LB Agar | + | + | − |
| LB Agar ⅓ | + | + | − |

+ Growth inhibition.
− No growth inhibition was shown

### Example 4

To quantitatively establish growth inhibition by the release of a diffusible bacteriocin-type molecule, a plate antagonism assay was performed using a dialysis membrane as mentioned above. The results showed a decrease of at least half the colony size compared to the assay without membrane (FIGS. 2a, b, c and d), observing a clear inhibition of the growth of the K2115 bacteria on the pathogenic bacteria under study. These results indicate that the molecule is less than 10 kDa in size, therefore, it crossed the dialysis membrane and diffused into the culture medium, generating inhibition of the growth of the pathogenic bacteria.

Based on the results of growth inhibition of pathogenic bacteria and in order to identify the genetic elements involved in the synthesis and secretion of the bacteriocin secreted by the K2115 bacteria, the bacterial genome was sequenced using the Illumina platform. Short and low coverage contigs were filtered, resulting in a set of 186 contigs, with an average coverage of 97× (N50=94,045 bp). The final assembly for the K2115 bacteria presented a total length of 6,645,031 bp (6.6 Mb) with a G+C content of 60.4% and 5996 ORFs (Table 6).

TABLE 6
Statistics of the sequencing and assembly of the K2I15 bacteria.

| Feature | Result | |
|---|---|---|
| Sequence size | 6660931 | |
| No contigs | 186 | |
| % GC | 60.4 | |
| Short contigs | 129 | bp |
| Long contigs | 452835 | bp |

| | |
|---|---|
| N50 | 94045 |
| No ORFs | 5996 |
| No ORFs with function | 4644 (77.5%) |
| Total No of RNAs | 71 |

The annotation of the genome sequence of the bacteria identified as Pseudomonas sp. strain K2115, was performed by the NCBI Prokaryotic Genome Automatic Annotation Pipeline (PGAAP), with accession number NIXO00000000 (Orellana et al., 2017), resulting in 5,996 coding sequences (CDS), 59 tRNAs, 104 pseudo genes and 7 rRNAs.

In order to identify in the genome sequence of BA K2115 putative clusters producing bacteriocin-type secondary metabolites, a bioinformatic analysis of the potential associated genes was performed using the AntiSmash bioinformatics platform, this allowed, by comparing the sequences with other known clusters (Weber et al., 2015), the identification of two clusters in the BA K2115 genome, which would contain candidate genes for the synthesis of bacteriocin-type proteins (cluster 1 and 8). These genes, as in the case of microcins, have a characteristic organization, consisting of a gene that encodes the microcin precursor, a gene that encodes the immunity protein against the bacteriocin itself, and one that encodes an export system (Kolter and Moreno, 1992).

Bioinformatics analysis of cluster 1 identified 10 ORFs (Open Reading Frames) in the K2115 BA genome. To identify the characteristic structure of a microcin-type bacteriocin within this cluster, the amino acid sequences derived from the different ORFs were compared with previously described bacteriocin sequences using the Blastp platform (NCBI). The analysis showed that the amino acid sequences mainly showed similarities with hypothetical proteins and not with previously described immunity proteins, microcins, or transporters. Therefore, the determination of a possible immunity protein and export system was based on the presence of 2 to 8 transmembrane regions, a characteristic of other described immunity proteins and transporters, such as those corresponding to microcins J25, H47, and V (Etayash et al., 2015). For this analysis, the bioinformatics tool Phobius Prediction was used, which identified six transmembrane regions in the amino acid sequence derived from ORF1_3 alone. Therefore, given this characteristic and its size, it could correspond to an immunity protein. However, none of these analyses identified an export system.

The bacteriocin candidate sequence would correspond to ORF1_6, because the derived amino acid sequence presents an AG motif characteristic of most of the described microcins, generating a residue of 65 amino acids that would correspond to the mature protein, with a leader peptide of 18 amino acids. However, the comparison made by multiple sequence alignment with the bioinformatics tool Clustal Omega, did not show similarity with sequences of other described bacteriocins, such as microcin E492 and microcin V. Therefore, the structural analysis of the genetic organization of cluster 1, showed a candidate sequence for a microcin-type bacteriocin, which is located close to a gene that could encode an immunity protein, but not to a gene that encodes an export system.

Bioinformatics analysis of cluster 8 using AntiSmash, identified 12 ORFs in the K2115 bacterial genome. To identify the structure corresponding to a microcin-type bacteriocin (immunity, microcin, and export system genes), the amino acid sequences derived from these ORFs were compared with sequences described in the GenBank database using Blastp at NCBI. This analysis showed that most of the ORFs showed similarities to hypothetical proteins, except for ORFs 33_34 and 33_35, which showed 100% similarity to ABC transporters. Therefore, both ORFs could correspond to ABC transporters involved in microcin export and are close to potential accessory proteins. The analysis was performed using the bioinformatics tool Phobius Prediction (as described above), allowing us to identify three transmembrane regions present in the amino acid sequence derived from ORF33_30, which would correspond to an immunity protein.

The amino acid sequence derived from ORF33_33 could correspond to a microcin-type bacteriocin, due to the presence of a GS motif characteristic of this type of protein, generating a residue of 82 amino acids that would correspond to the mature protein, with a leader peptide of 19 amino acids (Table 7). Interestingly, the structural analysis of this cluster showed that the candidate amino acid sequence for microcin, is close to genes that encode an export system, with their respective accessory proteins and a gene that encodes an immunity protein. Therefore, this genetic organization has the basic characteristics of a microcin, where the structure is made up of at least three types of genes: a gene that encodes the precursor of microcin, a gene that encodes the immunity protein against the bacteriocin itself and one that encodes the export system (Kolter and Moreno, 1992).

Physicochemical characteristics of ORF 1_6 and ORF33_33.

To determine the physicochemical characteristics of the bacteriocin candidate proteins (corresponding to ORF1_6 and ORF33_33, respectively), the amino acid sequences were analyzed using the bioinformatics platform ProtParam (Expasy.org). This platform provided characteristics associated with the primary structure of each protein, such as the number of amino acids, molecular weight, isoelectric point, stability index, aliphatic index, and half-life (Table 7).

Analysis of the results showed that the ORF1_6 protein is made up of 83 aa, with a molecular mass of 8.3 kDa. Sequence analysis of the mature protein (without the leader peptide sequence) presented 65 aa and a molecular mass of 6.6 kDa. The ORF33_33 protein is made up of 101 aa with a molecular mass of 9.9 kDa. Sequence analysis of the mature protein (without the leader peptide sequence) presented 82 aa and a molecular mass of 8.0 kDa (Table 7). Therefore, both proteins presented a mass less than 10 kDa, a relevant characteristic since it corresponds to the size described for a microcin-type bacteriocin.

TABLE 7
Main characteristics of the hypothetical candidate proteins for
bacteriocins. Analysis of the protein's primary structure
performed using the bioinformatics tool ProtParam (Expasy.org).

| | ORF1_6 protein | | ORF33_33 protein | |
|---|---|---|---|---|
| | Pre | | Pre | |
| Feature | Bacteriocin | Mature | Bacteriocin | Mature |
| Number of amino | 83 | 65 | 101 | 82 |
| acids | | | | |
| Molecular mass | 8316.51 | 6657.57 | 9983.32 | 8066.2 |
| | Dalton | Dalton | Dalton | Dalton |
| Isoelectric point | 8.59 | 8.26 | 8.8 | 7.89 |
| Instability index* | 12.77 | 19.46 | 22.1 | 18.3 |
| Aliphatic index | 56.63 | 45.08 | 68.02 | 56.34 |
| Half-life | >10 hours | >10 hours | >10 hours | >10 hours |

*Unstable protein: index with a value greater than 40.

Another important feature is the isoelectric point of 8.26 for the mature protein ORF1_6 and 7.89 for ORF33_33 (Table 7). The instability index indicated that both mature proteins are stable because they presented values lower than 40. The aliphatic index defines the relative volume occupied by the aliphatic side chains: alanine, valine, isoleucine and leucine, which can be interpreted as a positive factor for the increase in thermostability (Gill et al., 1989; Kite et al., 1982), a relevant condition similar to that obtained by the molecules that presented antimicrobial activity, because they were stable at 80° C. Finally, the half-life for all proteins was greater than 10 h, which refers to a stable protein.

The results of growth inhibition against other bacteria produced by the K2115 bacteria, together with the bioinformatics search in its genome for genetic elements that code for the synthesis of bacteriocins, allowed us to establish that cluster 1 and cluster 8 present in nodes 1 and 33 respectively, would contain the genetic elements involved in the synthesis and secretion of a bacteriocin-type molecule, specifically a microcin. Therefore, the sequences corresponding to ORF1_6 and ORF33_33 that would encode a microcin-type bacteriocin, were selected to be expressed in E. coli through a heterologous expression system called ProBac.

To verify that the recombinant proteins produced by the ORF1_6 and ORF33_33 genes are expressed and secreted, a plaque antimicrobial activity detection assay was performed on the indicator bacteria K. oxytoca and K. pneumoniae, where the initial culture was induced with 1 mM IPTG. The results showed that the heterologous expression system for the ORF1_6 gene synthesized a recombinant protein lacking antimicrobial activity. However, for the ORF33_33 gene, the expression system synthesized a protein with antimicrobial activity in both indicator strains, since a growth inhibition zone was observed (FIG. 3). This recombinant protein, obtained through the heterologous expression system, was called MicroPacin K2.

### Example 5

### Purification of the Recombinant Protein MicroPacina K2.

The recombinant protein MicroPacina K2 was purified from the supernatant of previously induced E. coli clone 33 using a Sep-Pack C18 hydrophobic column, by elution with a 10-100% methanol and water step gradient. To establish the elution profile of each fraction obtained from the column, the generation of growth inhibition zones on the lawn of the indicator bacteria K. oxytoca was determined (FIG. 4). MicroPacina K2 eluted mainly in the 80% and 90% methanol fractions, with 6400 AU/mL in the latter percentage of methanol.

In addition, the fractions that showed the greatest biological activity were determined by plate activity assays on the indicator pathogenic bacteria K. oxytoca and K. pneumoniae. The results showed that in fractions 80 and 90, a zone of growth inhibition was observed against both indicator bacteria, both on lawn (FIG. 5) and on soft agar (FIG. 6).

MicroPacina K2 protein eluted between the 80% and 90% methanol fractions, with an activity of 3200 AU/mL and 6400 AU/mL, respectively, demonstrating that it is a hydrophobic protein. The protein concentration was 34.2 mg/mL in the 80% fraction and 47.5 mg/mL in the 90% fraction. These results indicated that the specific activity was higher in the 90% fraction (Table 8). For this reason, purified MicroPacina K2 was used in the following assays.

TABLE 8
Activity of the purified fractions
of MicroPacina K2 by Sep-Pack C18.

| | | Concentration | Specific |
|---|---|---|---|
| Fraction | Activity | of protein | activity |
| (% methanol) | (UA/mL) | (mg/mL) | (AU/mg) |
| 80 | 3200 | 34.2 | 94 |
| 90 | 6400 | 47.5 | 134 |

SDS-PAGE was performed on the 90% fraction to verify the presence of the MicroPacin K2 protein. A protein band of approximately 8.3 kDa was observed (FIG. 7). These results are consistent with those previously reported using bioinformatics analysis.

To determine whether the in vitro antimicrobial effect of the MicroPacina K2 protein occurs through a bacteriostatic or bactericidal mechanism of action, a chromogenic plate analysis was performed on the reporter strain E. coli HB101. The MicroPacina K2 protein showed bactericidal activity (FIG. 8), since blue coloration was observed around the inhibition zone, this was due to the fact that the internal membrane of the reporter bacteria was damaged and, therefore, X-gal entered the bacteria. Since low molecular mass bacteriocins resistant to extreme conditions such as temperature, pH and solvents have been reported, the effect on the antimicrobial activity of MicroPacina K2 was determined.

To determine the effect of temperature, 100 mL of MicroPacin K2 with an activity of 6400 AU/mL was incubated for 1 h at different temperatures. The antimicrobial activity of MicroPacin K2 protein did not change under the effect of any of the temperatures at which it was incubated, demonstrating its thermostability (Table 9).

TABLE 9
Stability of MicroPacina K2 at different temperatures.

| Temperature | | Antimicrobial activity |
|---|---|---|
| (° C.) | Activity | UA/mL |
| −80 | + | 6400 |
| −20 | + | 6400 |
| 4 | + | 6400 |
| 18 | + | 6400 |
| 25 | + | 6400 |
| 37 | + | 6400 |
| 80 | + | 6400 |

To determine the effect of pH, the MicroPacin K2 protein was resuspended in buffer solutions with different pH values, as described in Materials and Methods. The recombinant MicroPacin K2 protein showed stability across the entire pH range tested, maintaining its activity at 6400 AU/mL (Table 10).

TABLE 10
Stability of MicroPacina K2 at different pH

| | | Antimicrobial activity |
|---|---|---|
| pH | Activity | UA/mL |
| 3 | + | 6400 |
| 4 | + | 6400 |
| 6 | + | 6400 |
| 7 | + | 6400 |
| 9 | + | 6400 |
| 10 | + | 6400 |

To determine the effect of solvents, the purified MicroPacin K2 protein was resuspended in different solvents, as described in Materials and Methods. The results showed that MicroPacin K2 protein activity remained stable against all treatments, except isopropanol (Table 11). The same solvents without the recombinant protein were used as a control.

TABLE 11
Stability of MicroPacina K2 to different solvents.

| | Antimicrobial activity |
|---|---|
| Solvent | (UA/mL) |
| Acetonitrile | 6400 |
| Ethanol | 6400 |
| Isopropanol | 0 |
| Dimethyl sulfoxide | 6400 |
| PBS | 6400 |
| H2O | 6400 |

To verify the protease stability of MicroPacina K2, the 90% fraction was treated with the proteolytic enzyme proteinase K for 2 h. The antimicrobial activity of MicroPacina K2 showed a decrease against this enzyme (Table 12), confirming the protein nature of this microcin-type bacteriocin.

TABLE 12
Protein nature of MicroPacina K2 (MPK2).

| | Antimicrobial activity |
|---|---|
| Treatment | (AU/mL) |
| MPK2 + Proteinase K | 800 |
| MPK2 | 6400 |
| MPK2 + Proteinase K inactivated | 6400 |

### Example 6

To determine the antimicrobial spectrum of action of MicroPacina K2, plate detection assays were performed on the pathogenic bacteria under study. MicroPacina K2 maintained the inhibition profile shown in the colony-based assays of the BA K2115 against the indicator bacteria K. oxytoca, K. pneumoniae, A. baumannii, S. boydii, S. sonnei and E. coli. However, MicroPacina K2 showed an inhibitory effect on the growth of the pathogenic bacteria E. coli O157:H7, V. parahaemolyticus, Enterobacter sp., S. enterica, S. flexneri, C. freundii, P. aeruginosa, demonstrating that MicroPacina K2 purified in the 90% fraction exerts an antimicrobial action on a broader spectrum of action of human pathogenic bacteria.

TABLE 13
Antimicrobial activity profile of the recombinant protein
MicroPacina K2 against human pathogenic bacteria.

| | | Supernatant | | |
|---|---|---|---|---|
| | MicroPacina | BA K2I15 | Positive | Negative |
| Pathogenic bacteria | K2 | concentrate | Control | Control |
| Escherichia coli ATCC 25922 | YES | YES | YES | NO |
| Salmonella Enteritidis ISP 1833/99 | NO | NO | YES | NO |
| Salmonella Typhi ISP2714/99 | NO | NO | YES | NO |
| Acinetobacter baumannii ISP287/04 | YES | YES | YES | NO |
| Klebsiella oxytoca ISP 1732/93 | YES | YES | YES | NO |
| Escherichia coli O157:H7 ISP 201/03 | YES | NO | YES | NO |
| Shigela boydii ISP 357/95 | YES | YES | YES | NO |
| Shigella sonnei ISP 2047/01 | YES | YES | YES | NO |
| Pseudomonas aeruginosa ATCC 27853 | NO | NO | YES | NO |
| Klebsiella pneumoniae ATCC 13883 | YES | YES | YES | NO |
| Staphylococcus aureus ATCC 25923 | NO | NO | YES | NO |
| Vibro parahaemolyticus RIMD 2210633 | YES | NO | YES | NO |
| Enterobacter sp. | YES | NO | YES | NO |
| Shigella flexneri | YES | NO | YES | NO |
| Citrobacter freundii | YES | NO | YES | NO |
| Morganella morgani | NO | NO | YES | NO |
| Pseudomonas aeruginosa O400 | YES | NO | YES | NO |

MicroPacina K2 inhibited growth. (YES)
MicroPacina K2 did NOT inhibit growth. (NO)

### Example 7

### Antibacterial Activity of Compositions Comprising MicroPacina K2.

Different compositions comprising the MicroPacin K2 polypeptide were prepared in different vehicles: 10% w/v methanol, 10% w/v glycerol, 0.5% w/v polyethylene glycol, and water. In the case of the MicroPacin K2 polypeptide, it was used at a concentration of 200 AU/mL in each composition.

A 5 μL aliquot of each of the prepared compositions was taken and added to a plate with Mueller Hinton medium containing the sensitivity indicator bacteria, previously seeded in the form of a lawn. The plates were then incubated for 24 hours, and the antibacterial activity of the compositions was observed according to the appearance and size of the inhibition zone. This test, in turn, was performed under the same conditions with the commercial bacteriocin Nisin as a comparator agent, which is considered a gold standard bacteriocin, for being approved by the FDA (Food and Drug Administration).

According to what was observed in the inhibition test, the compositions with MicroPacina, regardless of the vehicle used, are capable of inhibiting the growth of bacteria Staphylococcus aureus, Staphylococcus saprophyticus, Listeria monocytogenes, Echerichia coli 0157, Citrobacter freundii, Shigella sun (Table 14).

When comparing the results obtained for the compositions comprising the MicroPacina K2 polypeptide and those comprising nisin, it is observed that the compositions comprising MicroPacina can generate inhibition mainly for Gram-negative bacteria. It should be noted that in the case of the bacteria Escherichia coli O157:H7 and Shigella sonnei, nisin is not able to inhibit their growth (no inhibition halo is evident), while the compositions comprising the bacteriocin MicroPacina K2 effectively inhibit their growth.

TABLE 14
Antimicrobial activity of MicroPacina K2 (MPK2).

| | Zone of growth inhibition (mm) | | | | | | | |
|---|---|---|---|---|---|---|---|---|
| | Glycerol 10% | | PEG 0.5% | | Methanol 10% | | H₂O | |
| Bacteria | MPK2 | Nisin | MPK2 | Nisin | MPK2 | Nisin | MPK2 | Nisin |
| S. aureus | 0 | 9.0 ± | 0 | 8.0 ± | 0 | 8.0 ± | 0 | 9.0 ± |
| | | 0.01 | | 0.01 | | 0.5 | | 0.01 |
| S. | 0 | 10.0 ± | 0 | 9.0 ± | 0 | 10.0 ± | 0 | 10.0 ± |
| saprophyticus | | 0.5 | | 0.5 | | 0.5 | | 0.5 |
| B. cereus | 0 | 9.0 ± | 0 | 9.0 ± | 0 | 8.0 ± | 0 | 8.0 ± |
| | | 0.5 | | 0.5 | | 0.5 | | 0.5 |
| L. | 0 | 9.0 ± | 0 | 9.0 ± | 0 | 9.0 ± | 0 | 9.0 ± |
| monocytogenes | | 0.5 | | 0.5 | | 0.5 | | 0.01 |
| E. coli | 12.0 ± | 0 | 11.0 ± | 0 | 12.0 ± | 0 | 11.0 ± | 0 |
| O157:H7 | 0.4 | | 0.01 | | 0.01 | | 0.7 | |
| E. coli | 11.2 ± | 8.0 ± | 10.3 ± | 8.0 ± | 11.0 ± | 8.0 ± | 10.0 ± | 8.0 ± |
| DH5α | 0.6 | 0.5 | 0.5 | 0.5 | 0.01 | 0.5 | 0.5 | 0.5 |
| C. freundii | 9.2 ± | 8.0 ± | 8.8 ± | 7.0 ± | 8.7 ± | 8.0 ± | 7.6 ± | 8.0 ± |
| | 0.7 | 0.5 | 0.6 | 0.5 | 0.5 | 0.5 | 0.4 | 0.5 |
| S. sonnei | 11.0 ± | 0 | 11.0 ± | 0 | 11.6 ± | 0 | 11.4 ± | 0 |
| | 0.5 | | 0.4 | | 0.5 | | 0.01 | |

MPK2: MicroPacina K2

### Example 8

Antibacterial activity of compositions comprising MicroPacina K2 and an additional antibacterial agent.

In this example, the antibacterial effect of compositions comprising the polypeptide MicroPacin K2 and an additional antibacterial agent, including nisin (5,000 AU/mL), ampicillin (0.01 μg/mL), and tetracycline (0.05 μg/mL), is evaluated. The assay was performed using distilled water as the vehicle for the composition. The final concentration of MicroPacin K2 in the compositions evaluated was 200 AU/mL.

The results indicate that the combination of MicroPacina K2 with other antimicrobial agents inhibits the growth of the indicator strains tested (Table 15).

TABLE 15
Antimicrobial activity of MicroPacina K2 (MPK2)
in combination with other antimicrobial agents.

| | Mixtures | | | |
|---|---|---|---|---|
| | | MPK2 + | MPK2 + | MPK2 + |
| Bacteria | MP K2 | Nisin | Ampicillin | Tetracycline |
| S. aureus | − | + | + | + |
| S. saprophyticus | − | + | + | + |
| B. cereus | − | + | + | + |
| L. monocytogenes | − | + | + | + |
| E. coli O157:H7 | + | + | + | + |
| E. coli DH5α | + | + | + | + |
| C. freundii | + | + | + | + |
| S. sonnei | + | + | + | + |

MPK2: MicroPacina K2
+: Generation of growth inhibition
−: No growth inhibition

### CONCLUSIONS

The present invention focuses on the identification and characterization of a bacteria that secretes bacteriocin-type antimicrobial molecules, isolated from an extreme environment such as the Antarctic continent, specifically in the rhizosphere of Deschampsia antarctica, because this rhizospheric zone presents a peculiar bacterial diversity and competence (Texeira et al., 2010). In this aspect, the interaction that occurs between the soil and the roots of this plant allows the development of a complex microenvironment, which promotes the growth and diversity of microbial communities (Haldar et al., 2015) where microorganisms produce antimicrobial peptides that under natural conditions they use as an adaptation and competition mechanism against other microorganisms, inhibiting their growth and favoring their establishment in the bacterial ecosystem (Jenssen et al., 2006).

Since BA K2115 corresponds to an environmental bacterium that was isolated from the rhizosphere of D. antarctica in the Antarctic continent, there was no further background on its taxonomy. Therefore, this bacterium was characterized by phenotypic and molecular methods, in order to establish the metabolic and genetic characteristics that would allow its identification. The morphological, cellular and macroscopic characterization and the growth conditions of BA K2115 are consistent with those reported by Barrientos et al. (2008) and Berríos et al. (2012), who have described the presence of Gram-negative bacteria of the genus Pseudomonas isolated from cold environments, specifically from the rhizosphere of D. antarctica.

The protein deduced from ORF1_6 has a molecular mass of 6.6 kDa as a mature protein (without the leader peptide) and the protein deduced from ORF33_33 has a molecular mass of 8.0 kDa without the leader peptide sequence. Therefore, both proteins had a mass lower than 10 kDa, a relevant feature since it corresponds to the size described for a microcin-type bacteriocin (Corsini et al., 2010; Rebuffat, 2013) and agree with the results described in the antagonism experiments with dialysis membranes, where the size of the molecule with inhibitory effect was lower than 10 kDa.

Since microcin-type proteins have high hydrophobicity (Óscariz and Pisabarro, 2001) and solubility in organic solvents (Kolter and Moreno, 1992), MicroPacina K2 was purified by chromatography using Sep-Pack C18 hydrophobic columns (de Lorenzo, 1984). The protein eluted mainly in the 90% methanol-water fraction, thus, MicroPacina K2 has high hydrophobicity, and this correlates with what has been described in the literature for low molecular mass bacteriocins of the class II microcin-type (Baquero and Moreno, 1984). Similar results were obtained by Ferrer (2011) who described the elution of a microcin-type bacteriocin, isolated from P. aeruginosa 0400 in the 80% methanol-water fraction, which showed antimicrobial activity on pathogenic strains of Gram-positive bacteria.

Variations in temperature and pH did not affect the activity of purified MicroPacina K2, even considering that the buffer solutions used have a higher polarity index compared to methanol and therefore could have affected the structural conformation of microcin, which confirms what is described in the literature, where it is highlighted that most low molecular mass bacteriocins are resistant to extreme conditions, both pH and temperature (Héchard and Sahl., 2002; Guillor et al., 2004). Furthermore, these results allowed us to corroborate the results obtained through bioinformatics analysis, maintaining the characteristics previously described for the bacteriocin MicroPacina K2.

If we also consider that the recombinant protein MicroPacina K2 had a mass lower than 10 kDa, it would maintain the three characteristics described that are typical of microcin-type bacteriocins: low molecular mass molecules (lower than 10 kDa), resistance to pH and thermostability (Kolter and Moreno, 1992; Guillor et al., 2004; Rojas et al., 2014).

The use of an assay in chromogenic medium (Mardones and Venegas, 2000) allowed to establish that both the protein secreted by the BA and the recombinant protein MicroPacina K2 would exert their inhibitory action against pathogenic bacteria, through a bactericidal effect, which would indicate that its action target is probably located in the internal membrane of the target cell. This action mechanism has been described for other bacteriocins such as Ent35-MccV, which presents this type of bactericidal activity against enterohemorrhagic E. coli and Listeria monocytogenes (Acuña et al., 2012), as well as for microcin E492 (Lagos et al., 2001).

The results of plaque antagonism on pathogenic bacteria showed that both BA K2115 and MicroPacina K2 inhibited the growth of bacteria belonging to the Enterobacteriaceae family, such as K. oxytoca and K. pneumoniae, Shigella boydii, Shigella sonnei, E. coli O157:H7, among others. The inhibition of the growth of these species by MicroPacina K2 is relevant, because these bacteria are causative agents of diseases of clinical importance to humans, such as nosocomial infections, relevant both in Chile and worldwide (Hijazi et al., 2018; Tommasi et al., 2015; Pendleton et al., 2013; Llaca-Díaz et al., 2012; Livermore, D. 2012). Furthermore, these bacteria are resistant to the main antibiotics used today, a situation for which the medical and scientific community is on alert (Egan et al., 2017). Therefore, this new microcin-type bacteriocin produced and characterized in this invention represents a new alternative to face this scenario.

The present invention provides a new bioactive molecule with antimicrobial activity: a microcin-type bacteriocin called MicroPacina K2, obtained from a new bacteria identified as Pseudomonas sp. K2115 (Orellana et al., 2017) isolated from the rhizosphere of the Antarctic plant D. antarctica, for its potential use in biomedicine in the field of antimicrobials, which would make it possible to address the significant increase in bacterial resistance.

### DESCRIPTION OF FIGURES

FIG. 1.—Inhibition of the growth of human pathogenic bacteria by the BA K2115 in different culture media at 18° C., based on the antagonism test on lawn using the LB agar diffusion method. a) Colonies of BA on K. oxytoca lawn on ⅓ LB agar. b) Colonies on K. oxytoca lawn on Nut agar. c) Colonies on K. oxytoca lawn on LB agar. The grey arrow indicates the zone of inhibition of BA KI15.

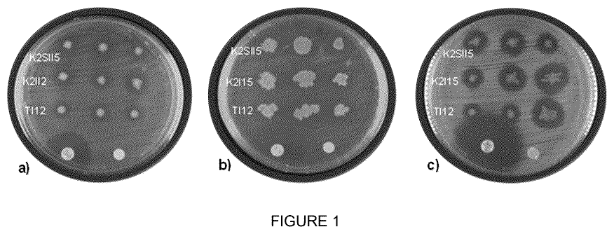

FIG. 2.—Inhibition of the growth of human pathogenic bacteria by BA K2115 using the membrane diffusion method. a) and c) A, B and C correspond to BA K2115 seeded in soft agar on a dialysis membrane and incubated at 4° C. (a) and 18° C. (c) for 24 h. Subsequently, the soft agar membrane was removed, and the different pathogenic bacteria were seeded on each LB agar plate and incubated at 37° C. for 24 h. As a control (D), soft agar without BA K2115 was used on a dialysis membrane. 1=K. pneumoniae, 2=K. oxytoca; 3=S. sonnei; 4=A. baumanni. A, B and C, replicates of each pathogenic bacteria. b) and d) Colony diameter (mm) of pathogenic bacteria K. pneumoniae, K. oxytoca, S. sonnei and A. baumannii by the effect of the diffusible bacteriocin-type molecule secreted by the BA K2115 at 4° C. (b) and 18° C. (c). p<0.05.

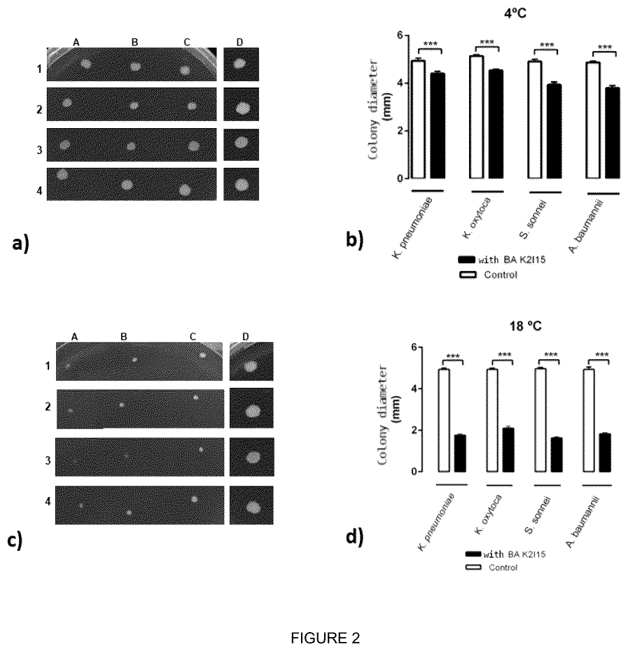

FIG. 3.—Detection of activity on plate with the recombinant protein MicroPacina K2 obtained by the heterologous expression system. Assay performed with 5 μL of unpurified supernatant of bacteria E. coli 33 against pathogenic bacteria K. pneumoniae (a) and K. oxytoca (b).

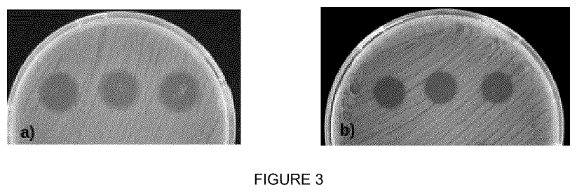

FIG. 4. Elution profile of the MicroPacin K2 protein using C18 column chromatography with a methanol step gradient. The left axis shows the tracking parameter, which corresponds to the antimicrobial activity in AU/mL (solid line), while the right axis shows the methanol concentration gradient used (dashed line).

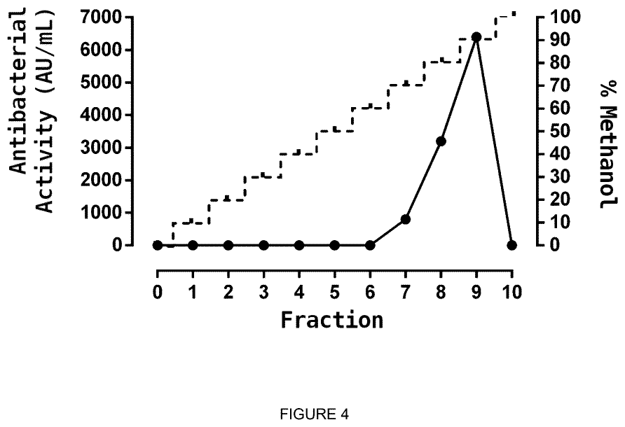

FIG. 5. Detection of plate activity of the MicroPacin K2 protein with the fractions purified by C18 column. Upper panel: lawn assay of the pathogenic bacteria K. pneumoniae. Lower panel: lawn assay of the pathogenic bacteria K. oxytoca. 80% and 90% represent the methanol fractions where the recombinant protein MicroPacina K2 eluted. Control +: Positive control. Control −: Negative control.

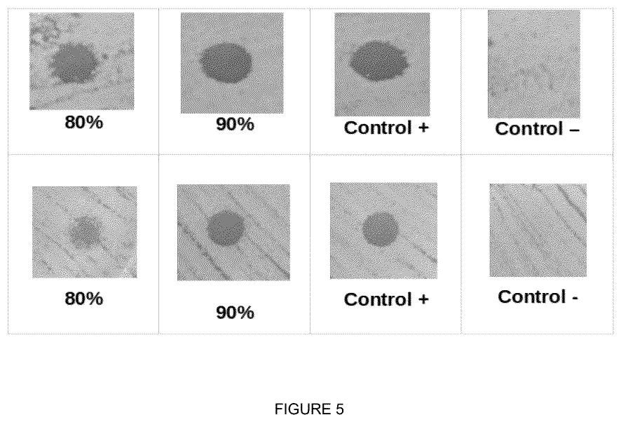

FIG. 6. Detection of plate activity of the protein MicroPacin K2 with the fractions purified by C18 column chromatography. Upper panel: soft agar assay of the pathogenic bacteria K. pneumoniae. Lower panel: soft agar assay of the pathogenic bacteria K. oxytoca. 80% and 90% represent the methanol fractions where the recombinant protein MicroPacina K2 eluted. Control +: Positive control. Control −: Negative control.

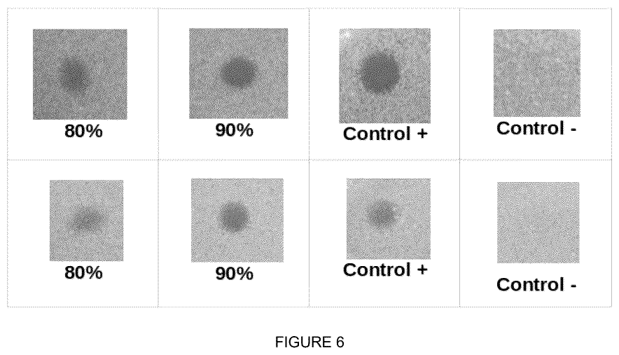

FIG. 7.—Polyacrylamide gel electrophoresis (SDS-PAGE) of purified MicroPacin K2. Lane 1, molecular mass standard (kDa); Lane 2, purified MicroPacin K2 by C18 column chromatography; Lane 3, E. coli 33 culture supernatant; Lane 4, negative control. 10, 15, 20, and 25 correspond to the molecular mass standard in kDa.

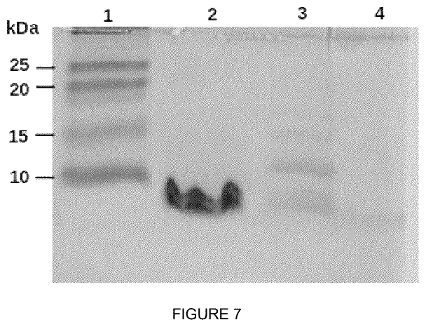

FIG. 8.—Antimicrobial effect of the MicroPacina K2 protein. Test performed using the Mardones and Venegas method (2000). The dark black color around the growth inhibition zone indicates a bactericidal effect of the antibacterial molecule. The white color around the growth inhibition zone indicates a bactericidal effect of the antibacterial molecule. a) Purified MicroPacina K2 presents a white color around the growth inhibition zone. b) Bactericidal control (Ampicillin). c) Bacteriostatic control (Tetracycline).

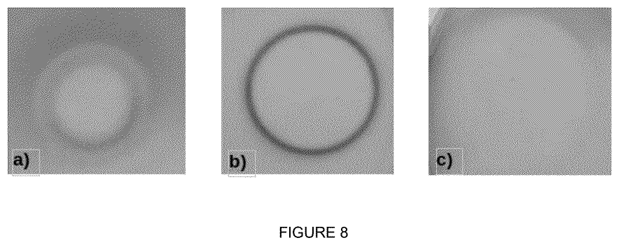

### BIBLIOGRAPHY

Baquero, F., Moreno, F. (1984). The microcins. FEMS Microbiology Letters; 23, 117-124. doi.org/10.1111/j.1574-6968.1984.tb01046.x
Beristain-Bauza, S. S., Palou, E., López-Malo, A. (2012). Bacteriocins: natural antimicrobials and their application in food. Selected Topics in Food Engineering, 6(2), 64-78.
Berríos, G., Cabrera, G., Gidekel, M., Gutiérrez-Moraga, A. (2012). Characterization of a novel Antarctic plant growth-promoting bacterial strain and its interaction with Antarctic hair grass (Deschampsia antarctica Desv.). Polar Biology, 36(3), 349-362. doi.org/10.1007/s00300-012-1264-6
Barrientos-Díaz, L., Gidekel, M., Gutiérrez-Moraga, A. (2008) Characterization of rhizospheric bacteria isolated from Deschampsia antarctica Desv. World Journal of Microbiology and Biotechnology, 24(10), 2289-2296. doi.org/10.1007/s11274-008-9743-1
Bulletin Chilean Antarctic. (2013). Chilean Antarctic Institute. Volume 32, No 1 and 2.
Boucher, H. W., Talbot, G. H., Bradley, J. S., Edwards, J. E., Gilbert, D., Rice, L. B., Scheld, M., Spellberg, B., Bartlett, J. (2009). Bad bugs, no drugs: no ESKAPE An update from the infectious diseases Society of America. Clinical Infectious Diseases, 48(1), 1-12. doi: 10.1086/595011.
Calisto, N., Navarro, L., Orellana, P., Wiesse, G., Gómez, C., Salazar, L., Cortés-Cortés, P., Gutierrez-Moraga, A., Gidekel, M., Corsini, G. (2021). Antibiotic resistance and antimicrobial activity of bacterial isolates from Antarctic soil. Annals of the Patagonian Institute 49: doi: 10.22352/AIP202149018.
Chen, L., Todd, R., Kiehlbauch, J., Walters, M., Kallen, A. (2017). Notes from the Field: Pan-Resistant New Delhi Metallo-Beta-Lactamase-Producing Klebsiella pneumoniae-Washoe County, Nevada, 2016. Morbidity and Mortality Weekly Report, 13; 66(1), 33. doi: 10.15585/mmwr.mm6601a7.
Clatworthy, A. E., Pierson, E., Hung, D. T. (2007). Targeting virulence: a new paradigm for antimicrobial therapy.
Nature Chemical Biology; 3(9), 541-548. doi: 10.1038/nchembio.2007.24
Corsini, G., Karahanian, E., Tello, M., Fernández, K., Rivero, D., Saavedra, J M., Ferrer, A. (2010). Purification and characterization of the antimicrobial peptide microcin N. FEMS Microbiology Letters, 312(2), 119-125. doi: 10.1111/j.1574-6968.2010.02106.x
Corsini, G., Baeza, M., Monasterio, O., Lagos, R. (2002). The expression of genes involved in microcin maturation regulates the production of active microcin E492. Biochemistry 84(5-6), 539-44. doi.org/10.1016/S0300-9084 (02) 01415-3
Cooper, M. A., & Shlaes, D. (2011).
Fix the antibiotics pipeline. Nature, 472, 32. doi.org/10.1038/472032a
Cotter, P. D., Ross, R. P., & Hill, C. (2013). Bacteriocins—a viable alternative to antibiotics? Nature Reviews Microbiology, 11, 95-105. doi: 10.1038/nrmicro2937
De Lorenzo, V., Martínez, J L, and Asensio, C. (1984). Microcin-mediated interactions between Klebsiella pneumoniae and Escherichia coli strains. Journal of General Microbiology, 130 (2), 391-400. doi: 10.1099/00221287-130-2-391
Donadio, S., Maffioli, S., Monciardini, P., Sosio, M., Jabes, D. (2010). Antibiotic discovery in the twenty-first century: current trends and future perspectives. Journal of Antibiotics, 63(8), 423-430. doi: 10.1038/ja.2010.62
Duquesne, S., Petit, V., Peduzzi, J., Rebuffat, S. (2007). Structural and functional diversity of microcins, gene-encoded antibacterial peptides from enterobacteria. Journal of Molecular Microbiology and Biotechnology, 13(4): 200-209. doi: 10.1159/000104748.
Etayash, H., Azmi, S., Dangeti, R., & Kaur, K. (2015). Peptide bacteriocins-structure activity relationships. Current Topics in Medicinal Chemistry, 16(2), 220-41. doi: 10.2174/1568026615666150812121103
Giddings, L A. and Newman, D. J. (2015). Bioactive Compounds from Terrestrial Extremophiles, Extremophilic Bacteria. Springer International Publishing, 90, 1-75. doi: 10.1007/978-3-319-13260-0
Guillor, O., Etzion, A., Riley, M A. (2008). The dual role of bacteriocins an anti- and probiotics. Advances in Applied Microbiology and Biotechnology, 81(4), 591-606. doi: 10.1007/s00253-008-1726-5
Guillor, O., Kirkup, B C, & Riley, M. A. (2004). Colicins and Microcins: The Next Generation Antimicrobials. Advances in applied microbiology, 54, 129-146. doi: 10.1016/S0065-2164 (04) 54005-4
Higerd, T B., Baechler, C A., Berk, R S. (1967). In vitro and in vivo characterization of pyocin. Journal Bacteriology, 93 (6), 1976-1986.
Hijazi, S., Visaggio, D., Pirolo, M., Frangipani, E., Bernstein, L., Visca, P. (2018). Antimicrobial Activity of Gallium Compounds on ESKAPE Pathogens. Frontier in Cellular and Infection Microbiology, 10(8), 316. doi: 10.3389/fcimb.2018.00316
Jenssen, H., Hamill, P., Hancock, R. (2006). Peptide antimicrobial agents. Clinical Microbiology Reviews, 19 (3), 491-511. doi: 10.1128/CMR.00056-05
Kolter, R. and Moreno, F. (1992). Genetics of ribosomally synthesized peptide antibiotics. Annual Review Microbiology, 46, 141-163. doi: 10.1146/annurev. e.46.100192.001041
Lagos, R., Baeza, M., Corsini, G., Hetz, C., Strahsburger, E., Castillo, J A., Vergara, C., Monasterio, O. (2001). Structure, organization and characterization of the gene cluster involved in the production of microcin E492, a channel-forming bacteriocin. Molecular Microbiology, 42(1), 229-43. doi.org/10.1046/j.1365-2958.2001.02630.x
Llaca-Díaz, J M., Mendoza-Olazarán, S., Camacho-Ortiz, A., Flores, S., Garza-González, E. (2012). One-year surveillance of ESKAPE pathogens in an intensive care unit of Monterrey, Mexico. Chemotherapy, 58(6), 475-481. doi: 10.1159/000346352.
Mardones, G., Venegas, A. (2000). Chromogenic plate assay distinguishing bacteriolytic from bacteriostatic activity of an antibiotic agent. Journal of Microbiology Methods, 40(3), 199-206. doi.org/10.1016/S0167-7012(00) 00125-1
Matthijs, S., Vander Wauven C., Cornu, B., Ye, L., Cornelis, P., Thomas, C M., Ongena, M. (2014). Antimicrobial properties of Pseudomonas strains producing the antibiotic mupirocin. Research in Microbiology, 165(8), 695-704. doi: 10.1016/j.resmic.2014.09.009
Michel-Briand, Y. and Baysse, C. (2002). The pyocins of Pseudomonas aeruginosa. Biochimie, 84 (56), 499-510. doi.org/10.1016/S0300-9084 (02) 01422-0
Nissen-Meyer, J. and Nes, IF. (1997). Ribosomally synthesized antimicrobial peptides: their function, structure, biogenesis, and mechanism of action. Archives of Microbiology, 167(2), 67-77.
Okami Y, Hotta K. (1988) Search and discovery of new antibiotics: Good fellow In Actinomycetes in Biotechnology. STM. Editors. Actinomycetes in Biotechnology. New York; Academic Press. Inc, 336. doi.org/10.1016/C2009-0-02828-1
Opazo, A., Lopes, B S., García, P., Domínguez Yévenes, M., Lima, C., Bello-Toledo, H., González-Rocha, G., Amyes, SGB. (2015). Draft genome sequence of a multidrug-resistant Acinetobacter baumannii strain from Chile. Genome Announcements, 3(4): e00687-15. doi: 10.1128/genomeA.00687-15.
Orellana, P., Pavón, A., Céspedes, S., Salazar, L., Gutiérrez, A., Castillo, D., Corsini, G. (2017). Draft genome sequence of Chilean Antarctic Pseudomonas sp. strain K2115. Genome Announcements, 5(33), e00771-17. doi: 10.1128/genomeA.00771-17
Orellana, P., Pavón, A., Calisto, N., Wiese, G., Navarro, N., Cortés-Cortés, P., Guidekel, M., Salazar, L., Gutierrez-Moraga, A., Corsini, G. (2022) Identification of Antarctic bacteria with antimicrobial activity isolated from the rhizosphere of Deschampsia Antarctica Desv. Annals of the Patagonian Institute 50:1-11. doi: 10.22352/AIP202250002.
Óscariz, J C. and Pisabarro, A G. (2001). Classification and mode of action of membrane-active bacteriocins produced by Gram-positive bacteria. International Microbiology, 4 (1), 13-19. doi: 10.1007/s101230100003
Padilla, C., Lobos, O., Brevis, P., Abaca, P., Hubert, E. (2002). Effects of the bacteriocin PsVP-10 produced by Pseudomonas sp. on sensitive bacterial strains. Latin American Journal of Microbiology, 44(1): 19-23.
Parret, A H. and De Mot, R. (2002). Bacteria killing their own kind: novel bacteriocins of Pseudomonas and other gamma-proteobacteria. Trends Microbiology, 10(3), 107-112.
Pendleton, J N., Gorman, sp., Gilmore, B. F. (2013). Clinical relevance of the ESKAPE pathogens. Expert Review of Anti-Infective Therapy, 11(3): 297-308. doi: 10.1586/eri.13.12.
Ravot, G J M Masson, F. Lefèvre. (2006). 34 applications of Extremophiles: the industrial screening of Extremophiles for valuable biomolecules. Methods of Microbiology, 35, 785-813.10.1016/S0580-9517(08)70037-0
Rebuffat, S. (2013). Microcins in action: amazing defense strategies or Enterobacteria. Biochemical Society Transactions, 40 (6), 1456-1462. doi: 10.1042/BST20120183.
Rennella, E., Sára, T., Juen, M., Wunderlich, C., Imbert, L., Solyom, Z., et al. (2017). RNA binding and chaperone activity of the E. coli cold-shock protein CspA. Nucleic Acids Research, 20; 45 (7), 4255-4268. doi: 10.1093/nar/gkx044.
Riley, M. A., & Wertz, J. E. (2002). Bacteriocins: evolution, ecology, and application. Annual Review of Microbiology, 56, 117-137. doi: 10.1146/annurev.micro.56.012302.161024
Riley, M., & Wertz, J. (2002). Bacteriocin diversity: ecological and evolutionary perspectives. Biochimie, 84 (3), 357-364. doi.org/10.1016/S0300-9084(02)01421-9.
Sánchez, L A., Gómez, F F., Delgado, O D. (2009). Cold-adapted microorganisms as a source of new antimicrobials. Extremophiles, 13 (1): 111-120. doi: 10.1007/s00792-008-0203-5.
Shlaes, D. M., Sahm, D., Opiela, C., Spellberg, B. (2013). The FDA reboot of antibiotic development. Antimicrobial Agents and Chemotherapy, 57(10), 4605-4607. doi: 10.1128/AAC.01277-13
Spiers, A. J., Buckling, A., Raincy, P. B. (2000).
The causes of Pseudomonas diversity. Microbiology, 146, 2345-2350. doi: 10.1099/00221287-146-10-2345.
Stoyanova, L., Ustyugova, E. and Netrusov, A. (2012). Antibacterial metabolites of lactic acid bacteria: their diversity and properties. Applied Biochemistry and Microbiology, 48(3), 259-275.
Tommasi, R., Brown, D G., Walkup, G K., Manchester, J I., Miller, A A. (2015). ESKAPEing the labyrinth of antibacterial discovery. Nature Reviews Drug Discovery, 14(8), 529-542. doi: 10.1038/nrd4572.

1. An antimicrobial polypeptide, corresponding to the amino acid sequence SEQ ID No 1, where said polypeptide has antimicrobial activity.

2. An antimicrobial composition comprising the polypeptide of claim 1 and a carrier.

3. The antimicrobial composition of claim 2, wherein a carrier is selected from: an organic solvent, aqueous solvent, non-aqueous solvent, a pH buffer and distilled water.

4. The antimicrobial composition of claim 2, wherein the polypeptide of amino acid sequence SEQ ID No 1 is present at a concentration of 100-2,000 AU/mL.

5. The antimicrobial composition of claim 4, wherein the polypeptide of amino acid sequence SEQ ID No 1 is present at a concentration of 200 AU/mL.

6. The antimicrobial composition of claim 2 further comprising a second antimicrobial agent, wherein the second antimicrobial agent is selected from the group of bacteriocins, beta-lactam antibiotics and tetracycline-type antibiotics.

7. Use of the antimicrobial composition of claim 2, wherein the antimicrobial composition serves to inhibit growth and proliferation of Gram-positive and Gram-negative bacteria.

8. The use of the antimicrobial composition of claim 7 wherein the use serves to inhibit the growth of bacteria selected from the group consisting of:
E. coli, E. coli O157:H7, Vibrio parahaemolyticus, Citrobacter freundii, Salmonella enterica, Shigella flexneri, Shigella boydii, Shigella sonnei, Klebsiella oxytoca, Klebsiella pneumoniae, Pseudomonas aeruginosa, Morganella morgani, Proteus mirabilis, Proteus vulgaris, Staphylococcus aureus, Staphylococcus epidermidis, Staphylococcus saprophyticus, Enterobacter sp., Streptococcus pneumoniae, Streptococcus pyogenes, Streptococcus mutans, Enterococcus faecium, Enterococcus faecalis, Listeria monocytogenes, Lactobacillus spp., Lactococcus spp., Bacillus cereus, Bacillus subtilis and Cutibacterium acnes among other Gram-negative and Gram-positive bacteria.

9. Use of the antimicrobial composition of claim 2, wherein the use serves in the preparation of a medicine useful in the treatment of conditions and/or diseases caused by Gram-positive and Gram-negative bacteria.

10. Use of the antimicrobial composition of claim 9, wherein the use serves in the preparation of a medicament useful in the treatment of bacteria selected from the group consisting of E. coli, E. coli 0157:H7, Vibrio parahaemolyticus, Citrobacter freundii, Salmonella enterica, Shigella flexneri, Shigella boydii, Shigella sonnei, Klebsiella oxytoca, Klebsiella pneumoniae, Pseudomonas aeruginosa, Morganella morgani, Proteus mirabilis, Proteus vulgaris, Staphylococcus aureus, Staphylococcus epidermidis, Staphylococcus saprophyticus, Enterobacter spp., Streptococcus pneumoniae, Streptococcus pyogenes, Streptococcus mutans, Enterococcus faecium, Enterococcus faecalis, Listeria monocytogenes, Lactobacillus spp., Lactococcus spp., Bacillus cereus, Bacillus subtilis and Cutibacterium acnes among other Gram-negative and Gram-positive bacteria.


SEQUENCE LISTING (1 sequence)

SEQ ID NO: 1 - protein, 82 aa, Pseudomonas sp. K2I15
ALSLSALTTTAHAADDMQKCFGVAEAGKNDCAAGAGTSCAGTSKVKDQANAWKLVPAGTCLKTPSATSPTGFGQEAAFTAKS